(19) United States
(12) Patent Application Publication
(10) Pub. No.: US 2022/0130495 A1
(43) Pub. Date: Apr. 28, 2022

(54) Method and Device for Determining Correlation Between Drug and Target, and Electronic Device

(71) Applicant: Beijing Baidu Netcom Science Technology Co., Ltd., Beijing (CN)

(72) Inventors: Shuangli LI, Beijing (CN); Jingbo ZHOU, Beijing (CN); Liang HUANG, Beijing (CN); Haoyi XIONG, Beijing (CN); Fan WANG, Beijing (CN); Tong XU, Beijing (CN); Hui XIONG, Beijing (CN); Dejing DOU, Beijing (CN)

(21) Appl. No.: 17/570,505

(22) Filed: Jan. 7, 2022

(30) Foreign Application Priority Data
Apr. 6, 2021 (CN) 202110367301.8

(51) Int. Cl. G16C 20/50; G16H 70/40

(52) U.S. Cl. CPC G16C 20/50; G16H 70/40

(57) ABSTRACT

A method for determining correlation between a drug and a target, and an electronic device are provided. The method includes: establishing a spatial molecular graph of a candidate drug and the target, the spatial molecular graph including an atomic node set and an edge set, the atomic node set including atoms in the candidate drug and atoms in the target, the edge set including at least one atom connection edge; inputting a first atom feature of the atomic node set and the spatial molecular graph into a first GAT for prediction, to obtain a second atom feature of the atomic node set; and determining a parameter value of the correlation between the candidate drug and the target in accordance with the second atom feature of the atomic node set.

### CROSS-REFERENCE TO RELATED APPLICATION

The present application claims a priority of the Chinese patent application No. 202110367301.8 filed in China on Apr. 6, 2021, the disclosure of which is incorporated herein by reference in its entirety.

### TECHNICAL FIELD

The present application relates to the field of a big data technology and a deep learning technology in a computer technology, in particular to a method and a device for determining correlation between a drug and a target, and an electronic device.

### BACKGROUND

For the research and development of a new drug, it is an important phase to predict binding affinity (also referred to as correlation) between the new drug and a target. In this phase, the affinity between a plurality of candidate new drugs and the target is measured and ranked, so as to find a new drug of real worth.

Currently, during the prediction, a Gaussian screening test is commonly adopted.

### SUMMARY

An object of the present application is to provide a method and a device for determining correlation between a drug and a target, and an electronic device+9−.

In one aspect, the present application provides in some embodiments a method for determining correlation between a drug and a target, including: establishing a spatial molecular graph of a candidate drug and the target, the spatial molecular graph including an atomic node set and an edge set, the atomic node set including atoms in the candidate drug and atoms in the target, the edge set including at least one atom connection edge; inputting a first atom feature of the atomic node set and the spatial molecular graph into a first Graph Attention Network (GAT) for prediction, so as to obtain a second atom feature of the atomic node set; and determining a parameter value of the correlation between the candidate drug and the target in accordance with the second atom feature of the atomic node set.

According to the method for determining the correlation between the drug and the target in the embodiments of the present application, the spatial molecular graph of the candidate drug and the target is established. Next, the first atom feature of the atomic node set and the spatial molecular graph into the first GAT for prediction, i.e., the prediction is performed using the first GAT to obtain the second atom feature of the atomic node set. Then, the parameter value of the correlation between the candidate drug and the target is determined in accordance with the second atom feature of the atomic node set. As a result, the prediction is performed without a Gaussian screening test, so it is able to reduce a computational burden, and determine the correlation between the drug and the target efficiently.

In another aspect, the present application provides in some embodiments a device for determining correlation between a drug and a target, including: an establishment module configured to establish a spatial molecular graph of a candidate drug and the target, the spatial molecular graph including an atomic node set and an edge set, the atomic node set including atoms in the candidate drug and atoms in the target, the edge set including at least one atom connection edge; a prediction module configured to input a first atom feature of the atomic node set and the spatial molecular graph into a first GAT for prediction, so as to obtain a second atom feature of the atomic node set; and a first determination module configured to determine a parameter value of the correlation between the candidate drug and the target in accordance with the second atom feature of the atomic node set.

In yet another aspect, the present application provides in some embodiments an electronic device, including at least one processor, and a memory in communication connection with the at least one processor and storing therein an instruction executed by the at least one processor. The instruction is executed by the at least one processor, so as to implement the method for determining the correlation between the drug and the target in the embodiments of the present application.

In still yet another aspect, the present application provides in some embodiments a non-transient computer-readable storage medium storing therein a computer instruction. The computer instruction is executed by a computer so as to implement the above-mentioned method for determining the correlation between the drug and the target in the embodiments of the present application.

In still yet another aspect, the present application provides in some embodiments a computer program product including a computer program. The computer program is executed by a processor so as to implement the above-mentioned method for determining the correlation between the drug and the target in the embodiments of the present application.

### BRIEF DESCRIPTION OF THE DRAWINGS

The following drawings are provided to facilitate the understanding of the present application, but shall not be construed as limiting the present application. In these drawings,

FIG. 1 is a flow chart of a method for determining correlation between a drug and a target according to an embodiment of the present application;

FIG. 2 is a schematic view showing a principle of range encoding in the method for determining the correlation between the drug and the target according to an embodiment of the present application;

FIG. 3 is a schematic view showing a principle of the method for determining the correlation between the drug and the target according to an embodiment of the present application;

FIG. 4 is a schematic view showing a device for determining correlation between a drug and a target according to an embodiment of the present application; and

FIG. 5 is a block diagram of an electronic device for implementing the method for determining the correlation between the drug and the target according to an embodiment of the present application.

### DETAILED DESCRIPTION

In the following description, numerous details of the embodiments of the present application, which should be deemed merely as exemplary, are set forth with reference to accompanying drawings to provide understanding of the embodiments of the present application. Therefore, those skilled in the art will appreciate that modifications or replacements may be made in the described embodiments without departing from the scope and spirit of the present application. Further, for clarity and conciseness, descriptions of known functions and structures are omitted.

As shown in FIG. 1, the present application provides in some embodiments a method for determining correlation between a drug and a target, which includes the following steps.

Step S101: establishing a spatial molecular graph of a candidate drug and the target.

The spatial molecular graph includes an atomic node set and an edge set, the atomic node set includes atoms in the candidate drug and atoms in the target, and the edge set includes at least one atom connection edge.

The candidate drug is a compound consisting of a plurality of atoms. The target of the drug is a position where the drug and a body biomacromolecule are gathered, and it may also be understood as a protein. As an important part in a drug discovery process, the prediction of interaction between the drug and the target is represented by prediction of affinity between the drug and the target, and the correlation may be just understood as affinity.

In the embodiments of the present application, the spatial molecular graph of the candidate drug (compound) and the target (protein) is established at first. For example, the spatial molecular graph is represented by G=(V, E), where V represents the atomic node set, V=V_{M} ∪Vₚ={a₁, a₂, . . . a_{N}}, V_{M} represents an atom set of the candidate drug, V_{P} represents an atom set of the protein, aᵢ represents an i^{th} atomic node and 1≤i≤N, and E represents the edge set including at least one atom connection edge, i.e., an edge connecting at least one pair of atomic nodes. Any pair of atomic nodes include two atomic nodes. It should be appreciated that, there is the atom connection edge between any two atoms merely when the two atoms meet a certain condition, otherwise, there is no atom connection edge.

Step S102: inputting a first atom feature of the atomic node set and the spatial molecular graph into a first GAT for prediction, to obtain a second atom feature of the atomic node set.

The atomic node set includes a plurality of atomic nodes, so the first atom feature of the atomic node set includes a first atom feature of each atomic node in the plurality of atomic nodes. At first, the first atom feature of the atomic node set is obtained, and the first atom feature includes, but not limited to, an atom type, the quantity of neighboring nodes, and the distribution of chemical bonds. The quantity of neighboring nodes for a certain atomic node represents the quantity of nodes having chemical bonds with the atomic node. The distribution of the chemical bonds for a certain atomic node represents the distribution of the chemical bonds for the atomic node in a corresponding candidate drug or target. In the embodiments of the present application, the first atom feature of the atomic node set and the spatial molecular graph are inputted into the first GAT for prediction, and then the first GAT outputs the second atom feature of the atomic node set. The second atom feature includes a second atom feature of each atomic node in the atomic node set.

It should be appreciated that, in a Graph Convolutional Network (GCN), a topical graph structure and a node feature are combined to obtain a good effect in a node classification task. However, a combination mode of a neighboring node feature in the GCN depends on the graph structure, leading to a limitation on a generalization ability of the GCN on the other graph structure. In the GAT, weighted summation is performed on the neighboring node features using an attention mechanism, and a weight of each neighboring node feature depends on the node feature and is independent of the graph structure. In other words, in the GAT, a fixed, standardized operation in the GCN is replaced with the attention mechanism, so the generalization ability is relatively strong. In the embodiments of the present application, the second atom feature different from the first atom feature and capable of representing an atom feature is obtained through the GAT in accordance with the first atom feature and the spatial molecular graph, so as to improve the atom representation accuracy.

Step S103: determining a parameter value of the correlation between the candidate drug and the target in accordance with the second atom feature of the atomic node set.

The parameter value of the correlation between the candidate drug and the target is determined in accordance with the second atom feature of the atomic node set, so as to predict the affinity between the candidate drug and the target. The larger the parameter value, the stronger the affinity; the smaller the parameter value, the weaker the affinity.

According to the method for determining the correlation between the drug and the target in the embodiments of the present application, the spatial molecular graph of the candidate drug and the target is established. Next, the first atom feature of the atomic node set and the spatial molecular graph into the first GAT for prediction, i.e., the prediction is performed using the first GAT to obtain the second atom feature of the atomic node set. Then, the parameter value of the correlation between the candidate drug and the target is determined in accordance with the second atom feature of the atomic node set. As a result, the prediction is performed without a Gaussian screening test, so it is able to reduce a computational burden, and determine the correlation between the drug and the target efficiently.

In a possible embodiment of the present application, the second atom feature of the atomic node set is inputted into a fully connected layer, and the parameter value of the correlation between the candidate drug and the target is outputted by the fully connected layer.

In a possible embodiment of the present application, the establishing the spatial molecular graph of the candidate drug and the target includes establishing the spatial molecular graph in accordance with a distance between atomic nodes in the atomic node set. A distance between two atomic nodes for any edge in the edge set is smaller than or equal to a predetermined distance threshold.

A coordinate position of each atomic node in the atomic node set is obtained in advance in a three-dimensional space using a conventional method, which will not be particularly defined herein. A distance between any two atoms in the atomic node set in the three-dimensional space is calculated in advance to obtain a distance matrix D. The distance matrix D includes the distance between any two atomic nodes in the atomic node set, e.g., Dᵢⱼ represents a distance between an i^{th} atomic node and a j^{th} atomic node. Subsequently, an edge connecting the atomic nodes is determined in accordance with the predetermined distance threshold θ_{d} (e.g., 5 Å), and the edge set E is expressed as E={eᵢⱼ=(aᵢ, aⱼ)|aᵢ, aⱼ∈V, Dᵢⱼ≤θ_{d}}, where aᵢ represents an i^{th} atomic node in the atomic node set, aⱼ represents a j^{th} atomic node in the atomic node set, eᵢⱼ represents an edge connecting the i^{th} atomic node and the j^{th} atomic node, and 1≤j≤N. The distance between any two atomic nodes is smaller than or equal to the predetermined distance threshold, so an edge connecting the two atomic nodes may be established. It should be appreciated that, eᵢⱼ represents an edge connecting the i^{th} atomic node and the j^{th} atomic node with the i^{th} atomic node as an end point, i.e., the edge is a directed edge from the j^{th} atomic node to the i^{th} atomic node.

In an original molecule, a link between atoms is merely determined by a chemical bond, which is insufficient to model a relationship among the atoms in the molecule. In addition, there is no original chemical bond between the drug and the target. In order to obtain more complete correlation between the atoms, in the embodiments of the present application, the spatial molecular graph of the drug and the target is established in accordance with a spatial distance, and in the spatial molecular graph, the distance between the two atomic nodes for any edge in the edge set is smaller than or equal to the predetermined distance threshold. In this way, it is able to represent the correlation between the atoms in the drug and the atoms in the target in a better manner through the spatial molecular graph, thereby to improve the accuracy of the spatial molecular graph.

In a possible embodiment of the present application, prior to inputting the first atom feature of the atomic node set and the spatial molecular graph into the first GAT for prediction so as to obtain the second atom feature of the atomic node set, the method further includes: encoding the distance between the atomic nodes in the atomic node set to obtain a first distance vector between the atomic nodes in the atomic node set; and converting the first distance vector between the atomic nodes in the atomic node set into a target distance vector between the atomic nodes in the atomic node set.

The inputting the first atom feature of the atomic node set and the spatial molecular graph into the first GAT for prediction so as to obtain the second atom feature of the atomic node set includes: inputting the first atom feature of the atomic node set, the spatial molecular graph and the target distance vector between the atomic nodes in the atomic node set into the first GAT for prediction so as to obtain the second atom feature of the atomic node set.

The distance between the atomic nodes in the atomic node set may include a distance between any two atomic nodes in the atomic node set. In the embodiments of the present application, during the prediction of the correlation, the distance between the atomic nodes in the atomic node set is also taken into consideration. However, this distance is a scalar distance, i.e., a specific value, and it needs to be encoded to obtain a corresponding first distance vector. Different scalar distances correspond to different first distance vectors. The first distance vector may be understood as a sparse vector, and the first distance vector between the atomic nodes in the atomic node set may be converted into a dense vector, so as to obtain the target distance vector between the atomic nodes in the atomic node set, i.e., the obtained target distance vector is a dense vector. Then, the first atom feature of the atomic node set, the spatial molecular graph and the distance vector between the atomic nodes in the atomic node set are inputted into the first GAT for prediction, so as to obtain the second atom feature of the atomic node set. The parameter value of the correlation is determined in accordance with the second atom feature, so as to improve the accuracy of the parameter value of the correlation.

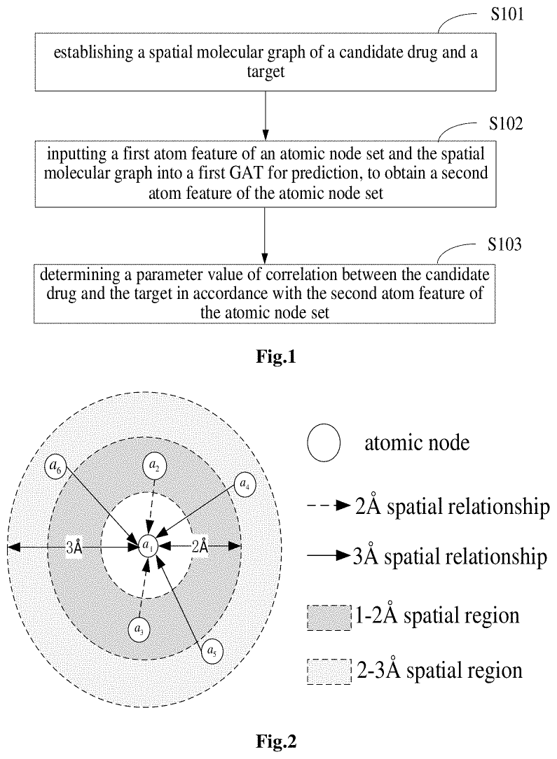

As an instance, the distance between the atomic nodes in the atomic node set is encoded through one-hot encoding, so as to obtain the distance vector between the atomic nodes in the atomic node set. In the one-hot encoding, a categorical vector is taken as a representation of a binary vector. At first, a categorical value (i.e., the distance in the embodiments of the present application) is mapped to an integral value, and each integral value is represented as a binary vector. Apart from an index of an integer, each integral value is a zero value and marked as 1. In the three-dimensional space, a position of each atomic node is defined through position coordinates (x, y, z), and the coordinates depend on a definition of a coordinate system (e.g., directions of axes x, y and z, and an origin of the coordinate). Hence, the distance is encoded in accordance with this relative position relationship. As shown in FIG. 2, a distance between a first atomic node a₁ and a second atomic node a₂ is within a range of (1 Å, 2 Å), i.e., greater than 1 Å and smaller than 2 Å; a distance between the first atomic node a₁ and a third atomic node a₃ is within a range of (1 Å, 2 Å); a distance between the first atomic node a₁ and a fourth atomic node a₄ is within a range of (2 Å, 3 Å); a distance between the first atomic node a₁ and a fifth atomic node a₅ is within a range of (2 Å, 3 Å); and a distance between the first atomic node a₁ and a sixth atomic node a₆ is within a range of (2 Å, 3 Å). A scalar distance between any pair of atomic nodes is encoded as a one-hot vector Dᵢⱼ^{R}, and Dᵢⱼ^{R} represents the first distance vector obtained by encoding the distance between the i^{th} atomic node and the j^{th} atomic node. Then, Dᵢⱼ^{R} is converted into a dense vector, so as to obtain a target distance vector pᵢⱼ between the i^{th} atomic node and the j^{th} atomic node. For example, Dᵢⱼ^{R} is converted using the following equation to obtain pᵢⱼ: pᵢⱼ=WₚDᵢⱼ^{R}, where Wₚ is a transfer matrix for converting the sparse vector into the dense vector.

In a possible embodiment of the present application, the inputting the first atom feature of the atomic node set, the spatial molecular graph, and the target distance vector between the atomic nodes in the atomic node set into the first GAT for prediction so as to obtain the second atom feature of the atomic node set includes: inputting the target distance vector between the atomic nodes in the atomic node set, the spatial molecular graph and the first atom feature of the atom node set into the first GAT for prediction, so as to obtain a target feature representation of each edge in the edge set; and predicting the first atom feature of the atomic node set, the target distance vector between the atomic nodes in the atomic node set and the target feature representation of the edge in the edge set in accordance with the first GAT, to obtain the second atom feature of the atomic node set.

During the determination of the second atom feature of the atomic nodes in the atomic node set, firstly edge nodes are aggregated to obtain the target feature representation of each edge in the edge set, and the edge node here refers to the edge in the edge set. A spatial distance depends on a pair of atomic nodes, and it is difficult for an existing neural network to effectively learn long-distance dependency during the aggregation. Hence, in the embodiments of the present application, distance information is aggregated into the edge node, and spatial structure information is captured through the propagation and aggregation of the edge nodes. One atom connection edge relates to one pair of atomic nodes, and after obtaining the target feature representation of the edge in the edge set, the first atom feature of the atomic nodes is updated through the aggregation of the atomic nodes in accordance with the target feature representation of the edge in the edge set, so as to obtain the second target atom feature.

In other words, in the embodiments of the present application, the target feature representation of the edge is determined at first, and during the determination of the target feature representation of the edge, the target distance vector between the atomic nodes in the atomic node set, the spatial molecular graph and the first atom feature of the atomic node set have been taken into consideration. Next, the second atom feature of the atomic node set is determined in accordance with the target feature representation of the edge in the edge set, i.e., during the determination of the second atom feature, not only the target feature representation of the edge but also the first atom feature of the atomic node set and the target distance vector between the atomic nodes in the atomic node set have been taken into consideration. In this regard, when determining the parameter value of the correlation in accordance with the second atom feature, it is able to improve the accuracy of determining the parameter value of the correlation.

In a possible embodiment of the present application, the inputting the target distance vector between the atomic nodes in the atomic node set, the spatial molecular graph and the first atom feature of the atomic node set into the first GAT for prediction so as to obtain the target feature representation of the edge in the edge set includes: determining a neighboring edge set for an edge between an i^{th} atomic node and a j^{th} atomic node in the edge set, where i and j are integers, 1≤i≤N, 1≤j≤M, N represents the total quantity of the atomic nodes in the atomic node set, and M represents the quantity of atomic nodes each having an edge with the i^{th} atomic node; determining an initial feature representation of the edge in the neighboring edge set in accordance with a target distance vector between atomic nodes for the edge in the neighboring edge set, a first atom feature of the atomic nodes for the edge in the neighboring edge set, as well as a first activation function, a first transfer matrix and an offset vector in the first GAT; determining a first standardized weight in accordance with the initial feature representation of the edge in the neighboring edge set, as well as a first weight matrix, a second activation function and a first attention weight in the first GAT; and determining a target feature representation of the edge between the i^{th} atomic node and the j^{th} atomic node in accordance with the initial feature representation of the edge in the neighboring edge set, the first standardized weight and the first weight matrix in the first GAT.

In the embodiments of the present application, the neighboring edge set for the edge between the i^{th} atomic node and the j^{th} atomic node may be understood as a neighboring edge set for the edge between the i^{th} atomic node and the j^{th} atomic node with the i^{th} atomic node as an end point, i.e., any edge in the neighboring edge set points to the i^{th} atomic node. For example, the spatial molecular graph G includes an edge eₖᵢ=(aₖ, aᵢ) and an edge eᵢⱼ=(aᵢ, aⱼ). The edge eₖᵢ is an edge between a k^{th} atomic node and the i^{th} atomic node with the i^{th} atomic node as an edge point, i.e., the edge eₖᵢ is an edge from the k^{th} atomic node to the i^{th} atomic node. The edge eₖᵢ is adjacent to the edge eᵢⱼ, so the edge eₖᵢ is a neighboring edge of the edge eᵢⱼ. In this way, it is able to determine all neighboring edges for the edge between the i^{th} atomic node and the j^{th} atomic node, thereby to obtain the neighboring edge set for the edge between the i^{th} atomic node and the j^{th} atomic node. The neighboring edge set for the edge between the i^{th} atomic node and the j^{th} atomic node includes all neighboring edges adjacent to the edge between the i^{th} atomic node and the j^{th} atomic node.

After determining the neighboring edge set for the edge between the i^{th} atomic node and the j^{th} atomic node, the initial feature representation of the edge in the neighboring edge set may be determined in accordance with the target distance vector between the atomic nodes for the edge in the neighboring edge set, the first atom feature of the atomic nodes for the edge in the neighboring edge set, the first activation function in the first GAT, the first transfer matrix in the first GAT, and the offset vector in the first GAT. It should be appreciated that, an initial feature representation of a target edge may be determined in accordance with a target distance vector between atomic nodes for a target edge in the neighboring edge set, a first atomic feature of two atomic nodes for the target edge, as well as the first activation function, the first transfer matrix and the offset vector in the first GAT. The target edge is any edge in the neighboring edge set. In other words, for each atom connection edge in the neighboring edge set, the initial feature representation of the target edge is determined in the above-mentioned way, so as to determine the initial feature representation of the edge in the neighboring edge set.

As an instance, for the target edge, the first atom feature of the two atomic nodes for the target edge is spliced with the target distance vector between the two atomic nodes for the target edge, so as to obtain a first splicing result. Next, the first transfer matrix is multiplied by the first splicing result to obtain a first target result. Next, the first target result is added to the offset vector to obtain a second target result. Then, the second target result is taken as an input of the first activation function, and the initial feature representation of the target edge is outputted through the first activation function.

As an instance, the initial feature representation e̅ₖ̅ᵢ̅ of the edge eₖᵢ between the k^{th} atomic node and the i^{th} atomic node is determined through e̅ₖ̅ᵢ̅=σ₁(Wₙₑ·[aₖ⁰⊕aᵢ⁰⊕pₖᵢ]+bₙₑ) where σ₁ represents the first activation function, Wₙₑ represents the first transfer matrix, aₖ⁰ represents the first atom feature of the k^{th} atomic node for the edge eₖᵢ, aᵢ⁰ represents the first atom feature of the i^{th} atomic node for the edge eₖᵢ, bₙₑ represents the offset vector, and pₖᵢ represents the target distance vector between the k^{th} atomic node and the i^{th} atomic node for the edge pₖᵢ. It should be appreciated that, e̅ₖ̅ᵢ̅=AGG_{node→edge}(aₖ⁰, aᵢ⁰, pₖᵢ).

As an instance, a_{k,i,j} is determined through

$${a}_{k , i , j} = \frac{exp ( {σ}_{2} ( {a}_{e}^{T} [ {W}_{e} \overline{{e}_{ij}} ⊕ {W}_{e} \overline{{e}_{ki}} ] ) )}{{\sum }_{{e}_{ti} ∈ {N}_{e} ( {e}_{ij} )} exp ( {σ}_{2} ( {a}_{e}^{T} [ {W}_{e} \overline{{e}_{ij}} ⊕ {W}_{e} \overline{{e}_{ti}} ] ) )} ,$$

where a_{k,i,j} is a first standardized weight related to the edge eₖᵢ and the edge eᵢⱼ and represents an importance level of the edge eₖᵢ relative to the edge eᵢⱼ during the determination of a target feature, σ₂ represents a second activation function, aₑ represents the first attention weight, Wₑ represents the first weight matrix, eᵤ represents an initial feature representation of the edge eᵤ, eₖ, represents the initial feature representation of the edge eₖᵢ in the neighboring edge set, e̅ₜ̅ᵢ̅ represents an initial feature representation of the edge eₜᵢ in the neighboring edge set, Nₑ(eᵢⱼ) represents a neighboring edge set for the edge eᵢⱼ, and Nₑ(eᵢⱼ)={eₖᵢ|eₖᵢ∈E, k≠j}.

As an instance, a target feature representation e̅ᵢ̅ⱼ̅ of the edge eᵢⱼ between the i^{th} atomic node and the j^{th} atomic node is determined through

$$\overline{\overline{{e}_{i j}}} = {\sum }_{{e}_{ki} ∈ {N}_{e} ( {e}_{ij} )} {a}_{k , i , j} {W}_{e} \overline{{e}_{k i}} .$$

It should be appreciated that, e̅ᵢ̅ⱼ̅=AGG_{edge→edge} (eᵢⱼ, Nₑ(eᵢⱼ)), where AGG represents aggregation.

Through the above process, the target feature representation of the edge between the i^{th} atomic node and the j^{th} atomic node in the edge set may be determined. 1≤i≤N and 1≤j≤M, so through the similar process, the target feature representation of each edge in the edge set is determined merely through updating values of i and j. When the values of i and j are updated, the neighboring edge set for the edge between the i^{th} atomic node and the j^{th} atomic node, the target distance vector between the i^{th} atomic node and the j^{th} atomic node, the first atom feature of the i^{th} atomic node and the first atom feature of the j^{th} atomic node are updated accordingly. In this way, it is able to obtain the target feature representation of the edge in the edge set.

In the embodiments of the present application, during the determination of the target feature representation, through the combination of the distance information, it is able to learn the distance dependency in the spatial molecular graph, and determine the second atom feature of the atomic node in accordance with the target feature representation of the edge, and then determine the parameter value of the correlation between the candidate drug and the target in accordance with the second atom feature. In this way, it is able to improve the accuracy of the parameter value of the correlation between the candidate drug and the target.

In a possible embodiment of the present application, the predicting the first atom feature of the atomic node set, the target distance vector between the atomic nodes in the atomic node set and the target feature representation of the edge in the edge set in accordance with the first GAT to obtain the second atom feature of the atomic node set includes: determining a target neighboring edge set for the i^{th} atomic node, an end point of any edge in the target neighboring edge set being the i^{th} atomic node; and determining the second atom feature of the i^{th} atomic node in accordance with a target feature representation of the edge in the target neighboring edge set, the first atom feature of the i^{th} atomic node, a target distance vector between atomic nodes for the edge in the target neighboring edge set as well as a second attention weight, a second transfer matrix and a second weight matrix in the first GAT.

Any edge in the target neighboring edge set points toward the i^{th} atomic node, and the second atom feature of the i^{th} atomic node may be determined through the above process. 1≤i≤N, so through the similar process, the second atom feature of each atomic node in the atomic node set is determined merely through updating a value of i. When the value of i is updated, the target neighboring edge set for the i^{th} atomic node, the target distance vector between the atomic nodes for the edge in the target neighboring edge set, the first atom feature of the i^{th} atomic node and the target distance vector between the atomic nodes in the edge in the target neighboring edge set are updated accordingly. In this way, it is able to obtain the target feature representation of each atomic node in the atomic node sets, i.e., the second atom feature of the atomic node set.

In the embodiments of the present application, during the determination of the second atom feature, through the combination of the distance information, it is able to learn the distance dependency in the spatial molecular graph, and take the target feature representation of the edge into consideration, and then determine the parameter value of the correlation between the candidate drug and the target in accordance with the second atom feature. In this way, it is able to improve the accuracy of the parameter value of the correlation between the candidate drug and the target.

As an instance, during the determination of the second atom feature of the i^{th} atomic node, at first the target feature representation of the edge in the target neighboring edge set may be converted to obtain a first conversion feature of the edge in the target neighboring edge set, e.g., h_{k,i,e}=Wₕe̅ₖ̅ᵢ̅, and then the first atom feature of the i^{th} atomic node may be converted to obtain a second conversion feature of the i^{th} atomic node, e.g., h_{i,a}=Wₕaᵢ⁰, where aᵢ⁰ represents the first atom feature of the i^{th} atomic node, Wₕ represents the second weight matrix, h_{k,i,e} represents the first conversion feature of the edge eₖᵢ, and h_{i,a} represents the second conversion feature of the i^{th} atomic node.

Next, an important level of an edge node is calculated with respect to different spatial distance relationships. An attention weight of the edge eₖᵢ relative to aᵢ is calculated through ωₖᵢ=σ₃ (aₙ^{T}[h_{i,a}⊕h_{k,i,j}⊕Wₛpₖᵢ]), where aₙ represents a second attention weight, Wₛ represents the second transfer matrix, and σ₃ represents a third activation function. Then, ωₖᵢ may be standardized, e.g., through a softmax function, so as to obtain a second standardized weight through

$${β}_{k i} = \frac{exp ( {ω}_{k i} )}{{\sum }_{{e}_{ki} ∈ {N}_{eon} ( {a}_{i} )} exp ( {ω}_{k i} )} ,$$

where βₖᵢ represents the second standardized weight after standardizing ωₖᵢ, and Nₑₒₙ(aᵢ) represents the target neighboring edge set for the i^{th} atomic node.

Finally, the atomic nodes are aggregated and updated in accordance with the second attention weight βₖᵢ, and the second atom feature a̅ᵢ̅ of the i^{th} atomic node aᵢ is determined through a̅ᵢ̅=σ₄(Σₑ_{ₖᵢ}_{∈N}_{ₑₒₙ}₍ₐ_{ᵢ}₎βₖᵢh_{k,i,e}).

In this way, the second atom feature of each atomic node in the atomic node set may be obtained. A sum of the second atom features of all the atomic nodes is obtained as a representation of the molecular graph

$$g = {\sum }_{i = 1}^{N} \overline{{a}_{i}} ,$$

and inputted into a fully connected layer consisting of a plurality of fully-connected layers cascaded to each other. The prediction of the affinity is performed through the fully-connected layer, so as to obtain the parameter value of the correlation, e.g., y̅=W₀MLP(g)+b₀, where y̅ represents the predicted parameter value of the correlation between the candidate drug and the target, MLP is a Multi-Layer Perceptron, W₀ represents a weight parameter matrix, and b₀ is an offset parameter.

In a possible embodiment of the present application, the first GAT may be a hierarchical GAT, i.e., it includes L layers of GATs, where L is an integer greater than 1. In two adjacent layers of GATs, an input of the latter includes an output of the former. An input of a first layer of GAT in the L layers of GATs includes the target distance vector between the atomic nodes in the atomic node set, the spatial molecular graph and the first atom feature of the atomic node set. An output of an l^{th} layer of GAT includes an l^{th}-layer atom feature of the atomic node set, where 1≤l≤L. An output of a last GAT, i.e., an L^{th} layer of GAT, includes an L^{th}-layer atom feature of the atomic node set, i.e., the second atom feature of the atomic node set. The l^{h}-layer atom feature is obtained by predicting an (l−1)^{th}-layer atom feature of the atomic node set, the target distance vector between the atomic nodes in the atomic node set and an l^{h}-layer target feature representation of the edge in the edge set according to the l^{h} layer of GAT in the first GAT, and the l^{h}-layer target feature representation of the edge in the edge set is obtained through inputting the target distance vector between the atomic nodes in the atomic node set, the spatial molecular graph and the (l−1)^{th}-layer atom feature of the atomic node set into the l^{th}-layer of GAT for prediction.

As an instance, an l^{th}-layer initial feature representation e̅ₖ̅ᵢ̅^̅{̅l̅}̅ of the edge eₖᵢ between the k^{th} atomic node and the i^{th} atomic node may be determined through e̅ₖ̅ᵢ̅^̅{̅l̅}̅=σ₁(Wₙₑ^{l}·[aₖ^{l-1}⊕aᵢ^{l-1}⊕pₖᵢ]+bₙₑ^{l}), where σ₁ represents the first activation function, Wₙₑ^{l} represents a first transfer matrix of the l^{h} layer of GAT, aₖ^{l-1} represents an (1-1)^{th}-layer atom feature of the k^{th} atomic node for the edge eₖᵢ, aᵢ^{l-1} represents an (1-1)^{th}-layer atom feature of the i^{th} atomic node for the edge eₖᵢ, bₙₑ^{l} represents an offset vector of the l^{th} layer of GAT, and pₖᵢ represents a target distance vector between the k^{th} atomic node and the i^{th} atomic node for the edge eₖᵢ. For example, the first activation function may be a ReLu function.

As an instance, a_{k,i,j}^{l} may be determined through

$${a}_{k , i , j}^{l} = \frac{exp ( {σ}_{2} ( {a}_{e , l}^{T} [ {W}_{e}^{l} \overline{{e}_{ij}^{l}} ⊕ {W}_{e}^{l} \overline{{e}_{ki}^{l}} ] ) )}{{\sum }_{{e}_{ti} ∈ {N}_{e} ( {e}_{ij} )} exp ( {σ}_{2} ( {a}_{e , l}^{T} [ {W}_{e}^{l} \overline{{e}_{ij}^{l}} ⊕ {W}_{e}^{l} \overline{{e}_{ti}^{l}} ] ) )} ,$$

where a_{k,i,j}^{l} is a standardized weight of the l^{th} layer of GAT in the first standardized weight related to the edge eₖᵢ and the edge eᵢⱼ and it represents an importance level of the edge eₖᵢ relative to the edge eᵢⱼ in the l^{th} layer of GAT during the aggregation, σ₂ represents the second activation function, a_{e,l} represents a first attention weight of the l^{th} layer of GAT, Wₑ^{l} represents a first weight matrix of the l^{th} layer of GAT, e̅ᵢ̅ⱼ̅^̅{̅l̅}̅ represents an initial feature representation of the edge eⱼⱼ in the l^{h} layer of GAT, e̅ₖ̅ᵢ̅^̅{̅l̅}̅ represents an initial feature representation of the edge eₖᵢ in the l^{th} layer of GAT in the neighboring edge set, and Nₑ (eᵢⱼ) represents the neighboring edge set for the edge eᵢⱼ. For example, the second activation function may be a LeakyReLu function.

As an instance, a target feature representation of the edge eᵢⱼ between the i^{th} atomic node and the j^{th} atomic node in the l^{th} layer of GAT, i.e., an l^{th}-layer atom feature e̅ᵢ̅ⱼ̅^̅{̅l̅}̅ of the edge eᵢⱼ between the i^{th} atomic node and the j^{th} atomic node, may be determined through

$$\overline{\overline{{e}_{i j}^{l}}} = {\sum }_{{e}_{ki} ∈ {N}_{e} ( {e}_{ij} )} {a}_{k , i , j}^{l} {W}_{e}^{l} \overline{{e}_{k i}^{l}} .$$

The target neighboring edge set Nₑₒₙ (aᵢ) for the i^{th} atomic node may be expressed as Nₑₒₙ(aᵢ)={eₖᵢ|eₖᵢ=(aₖ,aᵢ)∈E}.

Prior to the node aggregation, the representations of the atomic nodes and the edge nodes are uniformly transferred to a same vector space, i.e., h_{k,i,e}^{l}=Wₕ^{l}e̅ₖ̅ᵢ̅^̅{̅l̅}̅ and h_{i,a}^{l}=Wₕ^{l}a̅ᵢ̅^̅{̅l̅-̅1̅}̅, where aᵢ^{l-1} represents an (1-1)^{th}-layer atom feature of the i^{th} atomic node aᵢ, wₕ^{l} represents a second weight matrix of the l^{th} GAT, e̅ₖ̅ᵢ̅^̅{̅l̅}̅ represents a target feature representation of the edge e̅ₖ̅ᵢ̅^̅{̅l̅}̅ between the i^{th} atomic node and the j^{th} atomic node in the l^{th} GAT, d̅ᵢ̅^̅{̅l̅-̅1̅}̅ represents an (1-1)^{th}-layer atom feature of the i^{th} atomic node aᵢ, i.e., a second atom feature of the i^{th} atomic node aᵢ in the (1-1)^{th} GAT. In the case that l=1, l−1 is 0, and at this time, a̅ᵢ̅⁰̅ represents the first atom feature of the i^{th} atomic node.

Next, an important level of the edge node is calculated with respect to different spatial distance relationships. An attention weight of the edge eₖᵢ relative to aᵢ in the l^{th} GAT may be calculated through ωₖᵢ^{l}=σ₃ (a_{n,l}^{T}[h_{i,a}^{l}⊕h_{k,i,j}^{l}⊕Wₛ^{l}pₖᵢ]), where a_{n,l} represents a second attention weight of the l^{th} GAT, Wₛ^{l} represents a second transfer matrix of the l^{th} GAT, and σ₃ represents a third activation function. Then, ωₖᵢ^{l} is standardized through a softmax function, i.e.,

$${β}_{ki}^{l} = \frac{exp ( {ω}_{ki}^{l} )}{{\sum }_{{e}_{k} ∈ {N}_{eon} ( {a}_{i} )} exp ( {ω}_{ki}^{l} )} ,$$

where βₖᵢ^{l} represents a second standardized weight of wₖᵢ^{l} in the l^{th} layer of GAT after the standardization of ωₖᵢ^{l}, and Nₑₒₙ (aᵢ) represents the target neighboring edge set for the i^{th} atomic node.

Finally, the atomic nodes are aggregated and updated in accordance with the attention weight βₖᵢ^{l}, which is similar to extending the GAT to a multi-head GAT, and the resultant representations are averaged through

$$\overline{{a}_{i}^{l}} = {σ}_{4} ( \frac{1}{P} {\sum }_{m = 1}^{P} {\sum }_{{e}_{ki} ∈ {N}_{eon} ( {a}_{i} )} {β}_{k i}^{l , m} {h}_{k , i , e}^{l . m} ) ,$$

where a̅ᵢ̅^̅{̅l̅}̅ represents the second atom feature of the i^{th} atomic node aᵢ in the l^{th} GAT, i.e., the l^{th}-layer atom feature of the i^{th} atomic node aᵢ, P represents the quantity of the multi-head GATs, i.e., the first GAT is a P-head GAT each including L layers of network attention networks, σ₄ represents a fourth activation function, βₖᵢ^{l,m} represents a second standardized weight obtained after standardizing the attention weight ωₖᵢ^{l,m} of the edge eₖᵢ relative to aᵢ in an l^{th} GAT of an m^{th}-head GAT, and h_{k,i,e}^{l,m} represents the first conversion feature of the edge eₖᵢ in the l^{th} GAT of the m^{th}-head GAT. The L layers of graph attention layers for space perception are superimposed so as to effectively learn a topological structure of the molecular graph and the space distance information. In addition, a̅ᵢ̅^̅{̅L̅}̅ represents the second atom feature of the i^{th} atomic node aᵢ obtained through the first GAT.

At a final prediction stage, a sum of the second atom features of all the atomic nodes is obtained as a representation of the molecular graph

$$g = {\sum }_{i = 1}^{N} \overline{{a}_{i}^{L}} ,$$

and the affinity is predicted subsequently through a plurality of fully-connected layers, i.e., y̅=W₀MLP(g)+b₀.

It should be appreciated that, when training the GAT, a mean square error of a prediction result ŷ of a training sample and a really observed result y is taken as a training loss function, i.e.,

$$ℒ = \frac{1}{\vert 𝒟 \vert} {\sum }_{𝒟} {( y - \hat{y} )}^{2} ,$$

where [symbol] represents the training sample, and | [symbol] | represents the quantity of training samples.

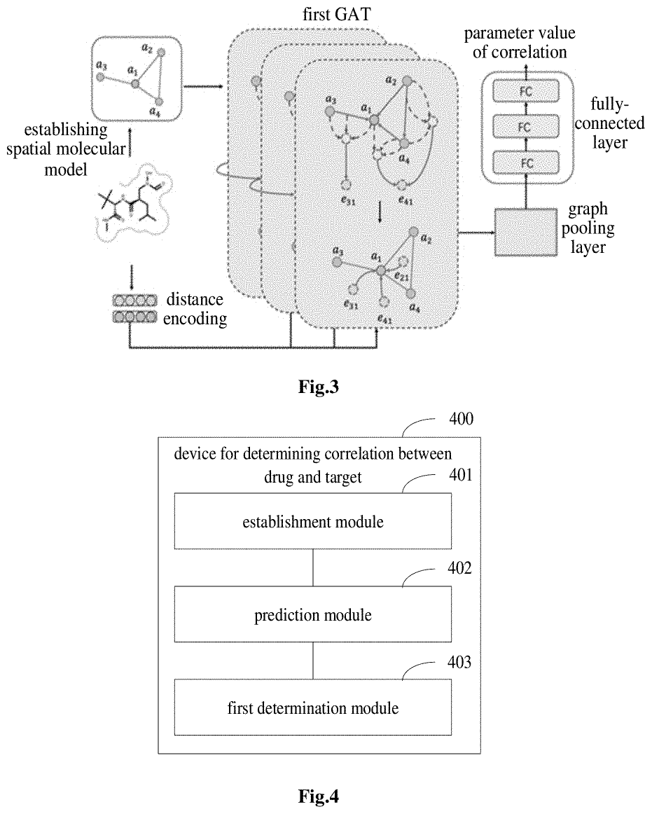

In the embodiments of the present application, as shown in FIG. 3, the molecular graph is established in accordance with a spatial relationship, and then a new model is proposed to learn the representation of a combination of the drug and the target in conjunction with space information. For the model, at first a plurality of layers of graph neural network modules is superimposed to update the representation of each atomic node, and each layer of graph neural network includes two parts, i.e., the learning of the aggregation of the atomic nodes and the learning of the aggregation of the edge nodes. Next, all the atomic nodes are aggregated by a graph pooling layer to obtain the representation of the molecular graph. Finally, the prediction is performed through a plurality of fully-connected layers.

In the embodiments of the present application, it is able to effectively learn distance information about each molecule in the three-dimensional space, thereby to rapidly, accurately predict the affinity of the combination of the drug and the target in conjunction with topological structure information about the molecular graph. To be specific, as compared with a traditional method and a physically based method, it is able to reduce a computational cost and a time cost. As compared with a machine learning method, it is unnecessary to extract features in accordance with domain expert knowledge, and it is able to improve the prediction accuracy of the model. In addition, as compared with a common deep learning model, it is able to accurately model the spatial association between the molecules, and learn the spatial distance information that cannot be learned by the traditional method, thereby to further improve the performance of the model.

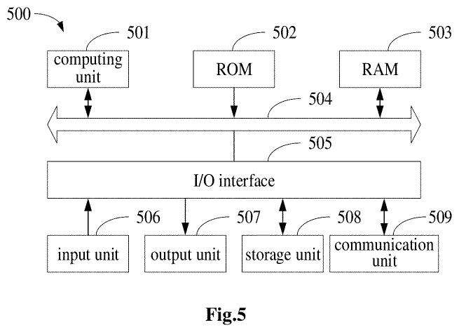

As shown in FIG. 4, the present application provides in some embodiments a device 400 for determining correlation between a drug and a target, which includes: an establishment module 401 configured to establish a spatial molecular graph of a candidate drug and the target, the spatial molecular graph including an atomic node set and an edge set, the atomic node set including atoms in the candidate drug and atoms in the target, the edge set including at least one atom connection edge; a prediction module 402 configured to input a first atom feature of the atomic node set and the spatial molecular graph into a first GAT for prediction, so as to obtain a second atom feature of the atomic node set; and a first determination module 403 configured to determine a parameter value of the correlation between the candidate drug and the target in accordance with the second atom feature of the atomic node set.

In a possible embodiment of the present application, the establishing the spatial molecular graph of the candidate drug and the target includes establishing the spatial molecular graph in accordance with a distance between atomic nodes in the atomic node set. A distance between two atomic nodes for any edge in the edge set is smaller than or equal to a predetermined distance threshold.

In a possible embodiment of the present application, the device further includes: an encoding module configured to encode the distance between the atomic nodes in the atomic node set to obtain a first distance vector between the atomic nodes in the atomic node set; and a first conversion module configured to convert the first distance vector between the atomic nodes in the atomic node set into a target distance vector between the atomic nodes in the atomic node set. The inputting the first atom feature of the atomic node set and the spatial molecular graph into the first GAT for prediction so as to obtain the second atom feature of the atomic node set includes inputting the first atom feature of the atomic node set, the spatial molecular graph and the target distance vector between the atomic nodes in the atomic node set into the first GAT for prediction so as to obtain the second atom feature of the atomic node set.

In a possible embodiment of the present application, the prediction module includes: a second determination module configured to input the target distance vector between the atomic nodes in the atomic node set, the spatial molecular graph and the first atom feature of the atom node set into the first GAT for prediction, so as to obtain a target feature representation of the edge in the edge set; and a third determination module configured to predict the first atom feature of the atomic node set, the target distance vector between the atomic nodes in the atomic node set and the target feature representation of the edge in the edge set according to the first GAT, to obtain the second atom feature in the atomic node set.

In a possible embodiment of the present application, the second determination module includes: a neighboring edge determination module configured to determine a neighboring edge set for an edge between an i^{th} atomic node and a j^{th} atomic node in the edge set, where i and j are integers, 1≤i≤N, 1≤j≤M, N represents the total quantity of the atomic nodes in the atomic node set, and M represents the quantity of atomic nodes each having an edge with the i^{th} atomic node; a first determination sub-module configured to determine an initial feature representation of the edge in the neighboring edge set in accordance with a target distance vector between atomic nodes for the edge in the neighboring edge set, a first atom feature of the atomic nodes for the edge in the neighboring edge set, as well as a first activation function, a first transfer matrix and an offset vector in the first GAT; a second determination sub-module configured to determine a first standardized weight in accordance with the initial feature representation of the edge in the neighboring edge set, as well as a first weight matrix, a second activation function and a first attention weight in the first GAT; and a third determination sub-module configured to determine a target feature representation of the edge between the i^{th} atomic node and the j^{th} atomic node in accordance with the initial feature representation of the edge in the neighboring edge set, the first standardized weight and the first weight matrix in the first GAT.

In a possible embodiment of the present application, the second determination module includes: a fourth determination sub-module configured to determine a target neighboring edge set for the i^{th} atomic node, an end point of any edge in the target neighboring edge set being the i^{th} atomic node; and a fifth determination sub-module configured to determine the second atom feature of the i^{th} atomic node in accordance with a target feature representation of the edge in the target neighboring edge set, the first atom feature of the i^{th} atomic node, a target distance vector between atomic nodes for the edge in the target neighboring edge set as well as a second attention weight, a second transfer matrix and a second weight matrix in the first GAT.

The device for determining the correlation between the drug and the target is used to implement the above-mentioned method with same technical features and technical effects, which will thus not be further particularly defined herein.

The present application further provides an electronic device, a computer-readable storage medium, and a computer program product.

In the embodiments of the present application, the non-transient computer-readable storage medium is configured to store therein computer instructions, and the computer instructions are executed by a computer to implement the above-mentioned method.

In the embodiments of the present application, the computer program product includes a computer program, and the computer program is executed by a computer to implement the above-mentioned method.

FIG. 5 is a schematic block diagram of the electronic device 500 for implementing the method in the embodiments of the present application. The electronic device is intended to represent all kinds of digital computers, such as a laptop computer, a desktop computer, a work station, a personal digital assistant, a server, a blade server, a main frame or any other suitable computers. The electronic device may also represent all kinds of mobile devices, such as a personal digital assistant, a cell phone, a smart phone, a wearable device and other similar computing devices. The components shown here, their connections and relationships, and their functions, are meant to be exemplary only, and are not meant to limit implementations of the present application described and/or claimed herein.

As shown in FIG. 5, the electronic device 500 includes a computing unit 501 configured to execute various appropriate actions and processings in accordance with computer programs stored in a Read Only Memory (ROM) 502 or computer programs loaded into a Random Access Memory (RAM) 503 via a storage unit 508. Various programs and data desired for the operation of the electronic device 500 may also be stored in the RAM 503. The computing unit 501, the ROM 502 and the RAM 503 may be connected to each other via a bus 504. In addition, an input/output (I/O) interface 505 may also be connected to the bus 504.

Multiple components in the electronic device 500 are connected to the I/O interface 505. The multiple components include: an input unit 506, e.g., a keyboard, a mouse and the like; an output unit 507, e.g., a variety of displays, loudspeakers, and the like; a storage unit 508, e.g., a magnetic disk, an optic disk and the like; and a communication unit 509, e.g., a network card, a modem, a wireless transceiver, and the like. The communication unit 509 allows the electronic device 500 to exchange information/data with other devices through a computer network and/or other telecommunication networks, such as the Internet.

The computing unit 501 may be any general purpose and/or special purpose processing components having a processing and computing capability. Some examples of the computing unit 501 include, but are not limited to: a central processing unit (CPU), a graphic processing unit (GPU), various special purpose artificial intelligence (AI) computing chips, various computing units running a machine learning model algorithm, a digital signal processor (DSP), and any suitable processor, controller, microcontroller, etc. The computing unit 501 carries out the aforementioned methods and processes, e.g., the method for determining the correlation between the drug and the target. For example, in some embodiments of the present application, the method may be implemented as a computer software program tangibly embodied in a machine readable medium such as the storage unit 508. In some embodiments of the present application, all or a part of the computer program may be loaded and/or installed on the electronic device 500 through the ROM 502 and/or the communication unit 509. When the computer program is loaded into the RAM 503 and executed by the computing unit 501, one or more steps of the foregoing method may be implemented. Optionally, in some other embodiments of the present application, the computing unit 501 may be configured in any other suitable manner (e.g., by means of firmware) to implement the above-mentioned method.

Various implementations of the aforementioned systems and techniques may be implemented in a digital electronic circuit system, an integrated circuit system, a field-programmable gate array (FPGA), an application specific integrated circuit (ASIC), an application specific standard product (ASSP), a system on a chip (SOC), a complex programmable logic device (CPLD), computer hardware, firmware, software, and/or a combination thereof. The various implementations may include an implementation in form of one or more computer programs. The one or more computer programs may be executed and/or interpreted on a programmable system including at least one programmable processor. The programmable processor may be a special purpose or general purpose programmable processor, may receive data and instructions from a storage system, at least one input device and at least one output device, and may transmit data and instructions to the storage system, the at least one input device and the at least one output device.

Program codes for implementing the methods of the present application may be written in one programming language or any combination of multiple programming languages. These program codes may be provided to a processor or controller of a general purpose computer, a special purpose computer, or other programmable data processing device, such that the functions/operations specified in the flow diagram and/or block diagram are implemented when the program codes are executed by the processor or controller. The program codes may be run entirely on a machine, run partially on the machine, run partially on the machine and partially on a remote machine as a standalone software package, or run entirely on the remote machine or server.

In the context of the present application, the machine readable medium may be a tangible medium, and may include or store a program used by an instruction execution system, device or apparatus, or a program used in conjunction with the instruction execution system, device or apparatus. The machine readable medium may be a machine readable signal medium or a machine readable storage medium. The machine readable medium includes, but is not limited to: an electronic, magnetic, optical, electromagnetic, infrared, or semiconductor system, device or apparatus, or any suitable combination thereof. A more specific example of the machine readable storage medium includes: an electrical connection based on one or more wires, a portable computer disk, a hard disk, a random access memory (RAM), a read only memory (ROM), an erasable programmable read only memory (EPROM or flash memory), an optic fiber, a portable compact disc read only memory (CD-ROM), an optical storage device, a magnetic storage device, or any suitable combination thereof.

To facilitate user interaction, the system and technique described herein may be implemented on a computer. The computer is provided with a display device (for example, a cathode ray tube (CRT) or liquid crystal display (LCD) monitor) for displaying information to a user, a keyboard and a pointing device (for example, a mouse or a track ball). The user may provide an input to the computer through the keyboard and the pointing device. Other kinds of devices may be provided for user interaction, for example, a feedback provided to the user may be any manner of sensory feedback (e.g., visual feedback, auditory feedback, or tactile feedback); and input from the user may be received by any means (including sound input, voice input, or tactile input).

The system and technique described herein may be implemented in a computing system that includes a back-end component (e.g., as a data server), or that includes a middle-ware component (e.g., an application server), or that includes a front-end component (e.g., a client computer having a graphical user interface or a Web browser through which a user can interact with an implementation of the system and technique), or any combination of such back-end, middleware, or front-end components. The components of the system can be interconnected by any form or medium of digital data communication (e.g., a communication network). Examples of communication networks include a local area network (LAN), a wide area network (WAN), Internet and a block chain network.

The computer system can include a client and a server. The client and server are generally remote from each other and typically interact through a communication network. The relationship of client and server arises by virtue of computer programs running on respective computers and having a client-server relationship to each other. The server may be a cloud server, also called as cloud computing server or cloud server, which is a host product in a cloud calculating service system, so as to overcome such defects as large management difficulty and insufficient service extensibility in a conventional physical host and a Virtual Private Server (VPS). The server may also be a server of a distributed system, or a server combined with block chain.

It should be appreciated that, all forms of processes shown above may be used, and steps thereof may be reordered, added or deleted. For example, as long as expected results of the technical solutions of the present application can be achieved, steps set forth in the present application may be performed in parallel, performed sequentially, or performed in a different order, and there is no limitation in this regard.

The above embodiments are for illustrative purposes only, but the present application is not limited thereto. It should be appreciated that the foregoing specific implementations do not constitute a limitation on the protection scope of the present application. A person skilled in the art should understand that various modifications, combinations, sub-combinations and substitutions can be made according to design requirements and other factors. Any modification, equivalent replacement and improvement made within the spirit and principle of the present application shall be included in the protection scope of the present application.

What is claimed is:

1. A method for determining a correlation between a candidate drug and a target, the method comprising:
establishing a spatial molecular graph of the candidate drug and the target, the spatial molecular graph comprising an atomic node set and an edge set, the atomic node set comprising atoms in the candidate drug and atoms in the target, the edge set comprising at least one atom connection edge;
inputting a first atom feature of the atomic node set and the spatial molecular graph into a first Graph Attention Network (GAT) for prediction to obtain a second atom feature of the atomic node set; and
determining a parameter value of the correlation between the candidate drug and the target in accordance with the second atom feature of the atomic node set.

2. The method according to claim 1, wherein establishing the spatial molecular graph of the candidate drug and the target comprises:
establishing the spatial molecular graph in accordance with a distance between atomic nodes in the atomic node set,
wherein a distance between two atomic nodes in the atomic node set for any edge in the edge set is smaller than or equal to a predetermined distance threshold.

3. The method according to claim 1, wherein prior to inputting the first atom feature of the atomic node set and the spatial molecular graph into the first GAT for prediction to obtain the second atom feature of the atomic node set, the method further comprises:
encoding a distance between atomic nodes in the atomic node set to obtain a first distance vector between the atomic nodes in the atomic node set; and
converting the first distance vector between the atomic nodes in the atomic node set into a target distance vector between the atomic nodes in the atomic node set,
wherein inputting the first atom feature of the atomic node set and the spatial molecular graph into the first GAT for prediction to obtain the second atom feature of the atomic node set comprises:
inputting the first atom feature of the atomic node set, the spatial molecular graph, and the target distance vector between the atomic nodes in the atomic node set into the first GAT for prediction to obtain the second atom feature of the atomic node set.

4. The method according to claim 3, wherein inputting the first atom feature of the atomic node set, the spatial molecular graph, and the target distance vector between the atomic nodes in the atomic node set into the first GAT for prediction to obtain the second atom feature of the atomic node set comprises:
inputting the target distance vector between the atomic nodes in the atomic node set, the spatial molecular graph, and the first atom feature of the atom node set into the first GAT for prediction, to obtain a target feature representation of an edge in the edge set; and
predicting the first atom feature of the atomic node set, the target distance vector between the atomic nodes in the atomic node set, and the target feature representation of the edge in the edge set in accordance with the first GAT to obtain the second atom feature of the atomic node set.

5. The method according to claim 4, wherein inputting the target distance vector between the atomic nodes in the atomic node set, the spatial molecular graph, and the first atom feature of the atomic node set into the first GAT for prediction to obtain the target feature representation of the edge in the edge set comprises:
determining a neighboring edge set for an edge between an i^{th} atomic node and a j^{th} atomic node in the edge set, where i and j are integers, 1≤i≤N, 1≤j≤M, N represents a total quantity of atomic nodes in the atomic node set, and M represents a quantity of atomic nodes in the atomic node set that have an edge with the i^{th} atomic node;
determining an initial feature representation of the edge in the neighboring edge set in accordance with a target distance vector between atomic nodes for the edge in the neighboring edge set, a first atom feature of the atomic nodes for the edge in the neighboring edge set, as well as a first activation function, a first transfer matrix, and an offset vector in the first GAT;
determining a first standardized weight in accordance with the initial feature representation of the edge in the neighboring edge set, as well as a first weight matrix, a second activation function, and a first attention weight in the first GAT; and
determining a target feature representation of the edge between the i^{th} atomic node and the j^{th} atomic node in accordance with the initial feature representation of the edge in the neighboring edge set, the first standardized weight, and the first weight matrix in the first GAT.

6. The method according to claim 5, wherein predicting the first atom feature of the atomic node set, the target distance vector between the atomic nodes in the atomic node set, and the target feature representation of the edge in the edge set in accordance with the first GAT to obtain the second atom feature of the atomic node set comprises:
determining a target neighboring edge set for the i^{th} atomic node, an end point of any edge in the target neighboring edge set being the i^{th} atomic node; and
determining the second atom feature of the i^{th} atomic node in accordance with a target feature representation of the edge in the target neighboring edge set, the first atom feature of the i^{th} atomic node, a target distance vector between atomic nodes for the edge in the target neighboring edge set, as well as a second attention weight, a second transfer matrix, and a second weight matrix in the first GAT.

7. The method according to claim 2, wherein prior to inputting the first atom feature of the atomic node set and the spatial molecular graph into the first GAT for prediction to obtain the second atom feature of the atomic node set, the method further comprises:
encoding a distance between atomic nodes in the atomic node set to obtain a first distance vector between the atomic nodes in the atomic node set; and
converting the first distance vector between the atomic nodes in the atomic node set into a target distance vector between the atomic nodes in the atomic node set,
wherein inputting the first atom feature of the atomic node set and the spatial molecular graph into the first GAT for prediction to obtain the second atom feature of the atomic node set comprises:
inputting the first atom feature of the atomic node set, the spatial molecular graph, and the target distance vector between the atomic nodes in the atomic node set into the first GAT for prediction to obtain the second atom feature of the atomic node set.

8. The method according to claim 7, wherein inputting the first atom feature of the atomic node set, the spatial molecular graph, and the target distance vector between the atomic nodes in the atomic node set into the first GAT for prediction to obtain the second atom feature of the atomic node set comprises:
inputting the target distance vector between the atomic nodes in the atomic node set, the spatial molecular graph, and the first atom feature of the atom node set into the first GAT for prediction to obtain a target feature representation of an edge in the edge set; and
predicting the first atom feature of the atomic node set, the target distance vector between the atomic nodes in the atomic node set, and the target feature representation of the edge in the edge set in accordance with the first GAT to obtain the second atom feature of the atomic node set.

9. An electronic device comprising:
at least one processor; and
a memory in communication connection with the at least one processor,
wherein the memory stores therein instructions capable of being executed by the at least one processor, wherein the at least one processor is configured to execute the instruction to implement steps of:
establishing a spatial molecular graph of a candidate drug and a target, the spatial molecular graph comprising an atomic node set and an edge set, the atomic node set comprising atoms in the candidate drug and atoms in the target, the edge set comprising at least one atom connection edge;
inputting a first atom feature of the atomic node set and the spatial molecular graph into a first Graph Attention Network (GAT) for prediction to obtain a second atom feature of the atomic node set; and
determining a parameter value of a correlation between the candidate drug and the target in accordance with the second atom feature of the atomic node set.

10. The electronic device according to claim 9, wherein establishing the spatial molecular graph of the candidate drug and the target comprises:
establishing the spatial molecular graph in accordance with a distance between atomic nodes in the atomic node set,
wherein a distance between two atomic nodes in the atomic node set for any edge in the edge set is smaller than or equal to a predetermined distance threshold.

11. The electronic device according to claim 9, wherein the at least one processor is further configured to execute the instruction to implement steps of, prior to inputting the first atom feature of the atomic node set and the spatial molecular graph into the first GAT for prediction to obtain the second atom feature of the atomic node set:
encoding a distance between atomic nodes in the atomic node set to obtain a first distance vector between the atomic nodes in the atomic node set; and
converting the first distance vector between the atomic nodes in the atomic node set into a target distance vector between the atomic nodes in the atomic node set,
wherein inputting the first atom feature of the atomic node set and the spatial molecular graph into the first GAT for prediction to obtain the second atom feature of the atomic node set comprises:
inputting the first atom feature of the atomic node set, the spatial molecular graph, and the target distance vector between the atomic nodes in the atomic node set into the first GAT for prediction to obtain the second atom feature of the atomic node set.

12. The electronic device according to claim 11, wherein inputting the first atom feature of the atomic node set, the spatial molecular graph and the target distance vector between the atomic nodes in the atomic node set into the first GAT for prediction to obtain the second atom feature of the atomic node set comprises:
inputting the target distance vector between the atomic nodes in the atomic node set, the spatial molecular graph and the first atom feature of the atom node set into the first GAT for prediction, to obtain a target feature representation of an edge in the edge set; and
predicting the first atom feature of the atomic node set, the target distance vector between the atomic nodes in the atomic node set, and the target feature representation of the edge in the edge set in accordance with the first GAT to obtain the second atom feature of the atomic node set.

13. The electronic device according to claim 12, wherein inputting the target distance vector between the atomic nodes in the atomic node set, the spatial molecular graph and the first atom feature of the atomic node set into the first GAT for prediction to obtain the target feature representation of the edge in the edge set comprises:
determining a neighboring edge set for an edge between an i^{th} atomic node and a j^{th} atomic node in the edge set, where i and j are integers, 1≤i≤N, 1≤j≤M, N represents a total quantity of atomic nodes in the atomic node set, and M represents a quantity of atomic nodes in the atomic node set that have an edge with the i^{th} atomic node;
determining an initial feature representation of the edge in the neighboring edge set in accordance with a target distance vector between atomic nodes for the edge in the neighboring edge set, a first atom feature of the atomic nodes for the edge in the neighboring edge set, as well as a first activation function, a first transfer matrix and an offset vector in the first GAT;
determining a first standardized weight in accordance with the initial feature representation of the edge in the neighboring edge set, as well as a first weight matrix, a second activation function, and a first attention weight in the first GAT; and
determining a target feature representation of the edge between the i^{th} atomic node and the j^{th} atomic node in accordance with the initial feature representation of the edge in the neighboring edge set, the first standardized weight, and the first weight matrix in the first GAT.

14. The electronic device according to claim 13, wherein predicting the first atom feature of the atomic node set, the target distance vector between the atomic nodes in the atomic node set, and the target feature representation of the edge in the edge set in accordance with the first GAT to obtain the second atom feature of the atomic node set comprises:
determining a target neighboring edge set for the i^{th} atomic node, an end point of any edge in the target neighboring edge set being the i^{th} atomic node; and
determining the second atom feature of the i^{th} atomic node in accordance with a target feature representation of the edge in the target neighboring edge set, the first atom feature of the i^{th} atomic node, a target distance vector between atomic nodes for the edge in the target neighboring edge set, as well as a second attention weight, a second transfer matrix, and a second weight matrix in the first GAT.

15. A non-transitory computer-readable storage medium storing therein computer instructions, wherein the computer instructions are configured to be executed by a computer to implement steps of:
establishing a spatial molecular graph of a candidate drug and a target, the spatial molecular graph comprising an atomic node set and an edge set, the atomic node set comprising atoms in the candidate drug and atoms in the target, the edge set comprising at least one atom connection edge;
inputting a first atom feature of the atomic node set and the spatial molecular graph into a first Graphical Attention Network (GAT) for prediction to obtain a second atom feature of the atomic node set; and
determining a parameter value of a correlation between the candidate drug and the target in accordance with the second atom feature of the atomic node set.

16. The non-transient computer-readable storage medium according to claim 15, wherein establishing the spatial molecular graph of the candidate drug and the target comprises:
establishing the spatial molecular graph in accordance with a distance between atomic nodes in the atomic node set,
wherein a distance between two atomic nodes in the atomic node set for any edge in the edge set is smaller than or equal to a predetermined distance threshold.

17. The non-transient computer-readable storage medium according to claim 15, wherein the computer instructions are further configured to be executed by a computer to implement steps of, prior to inputting the first atom feature of the atomic node set and the spatial molecular graph into the first GAT for prediction to obtain the second atom feature of the atomic node set:
encoding a distance between atomic nodes in the atomic node set to obtain a first distance vector between the atomic nodes in the atomic node set; and
converting the first distance vector between the atomic nodes in the atomic node set into a target distance vector between the atomic nodes in the atomic node set,
wherein inputting the first atom feature of the atomic node set and the spatial molecular graph into the first GAT for prediction to obtain the second atom feature of the atomic node set comprises:
inputting the first atom feature of the atomic node set, the spatial molecular graph and the target distance vector between the atomic nodes in the atomic node set into the first GAT for prediction to obtain the second atom feature of the atomic node set.

18. The non-transient computer-readable storage medium according to claim 17, wherein inputting the first atom feature of the atomic node set, the spatial molecular graph and the target distance vector between the atomic nodes in the atomic node set into the first GAT for prediction to obtain the second atom feature of the atomic node set comprises:
inputting the target distance vector between the atomic nodes in the atomic node set, the spatial molecular graph and the first atom feature of the atom node set into the first GAT for prediction, to obtain a target feature representation of an edge in the edge set; and
predicting the first atom feature of the atomic node set, the target distance vector between the atomic nodes in the atomic node set and the target feature representation of the edge in the edge set in accordance with the first GAT, to obtain the second atom feature of the atomic node set.

19. The non-transient computer-readable storage medium according to claim 18, wherein inputting the target distance vector between the atomic nodes in the atomic node set, the spatial molecular graph and the first atom feature of the atomic node set into the first GAT for prediction to obtain the target feature representation of the edge in the edge set comprises:
determining a neighboring edge set for an edge between an i^{th} atomic node and a j^{th} atomic node in the edge set, where i and j are integers, 1≤i≤N, 1≤j≤M, N represents a total quantity of atomic nodes in the atomic node set, and M represents a quantity of atomic nodes in the atomic node set that have an edge with the i^{th} atomic node;
determining an initial feature representation of the edge in the neighboring edge set in accordance with a target distance vector between atomic nodes for the edge in the neighboring edge set, a first atom feature of the atomic nodes for the edge in the neighboring edge set, as well as a first activation function, a first transfer matrix and an offset vector in the first GAT;
determining a first standardized weight in accordance with the initial feature representation of the edge in the neighboring edge set, as well as a first weight matrix, a second activation function and a first attention weight in the first GAT; and
determining a target feature representation of the edge between the i^{th} atomic node and the j^{th} atomic node in accordance with the initial feature representation of the edge in the neighboring edge set, the first standardized weight, and the first weight matrix in the first GAT.

20. The non-transient computer-readable storage medium according to claim 19, wherein predicting the first atom feature of the atomic node set, the target distance vector between the atomic nodes in the atomic node set and the target feature representation of the edge in the edge set in accordance with the first GAT to obtain the second atom feature of the atomic node set comprises:
determining a target neighboring edge set for the i^{th} atomic node, an end point of any edge in the target neighboring edge set being the i^{th} atomic node; and
determining the second atom feature of the i^{th} atomic node in accordance with a target feature representation of the edge in the target neighboring edge set, the first atom feature of the i^{th} atomic node, a target distance vector between atomic nodes for the edge in the target neighboring edge set, as well as a second attention weight, a second transfer matrix and a second weight matrix in the first GAT.
